(19) United States
(12) Patent Application Publication
(10) Pub. No.: US 2013/0178791 A1
(43) Pub. Date: Jul. 11, 2013

(54) Method and system for detecting and treating biological and chemical warfare agents

(71) Applicant: Jonathan C. Javitt, Chevy Chase, MD (US)

(72) Inventor: Jonathan C. Javitt, Chevy Chase, MD (US)

(21) Appl. No.: 13/345,771

(22) Filed: Jan. 9, 2012

(51) Int. Cl. A61M 5/168

(52) U.S. Cl. 604/66; 604/65; 604/67; 604/500; 604/503

(57) ABSTRACT

The present invention generally relates to radio-enabled devices for detection and remedy of biological and chemical warfare attack. More particularly, this invention relates to an on-body apparatus that provides a means of treatment which is innocuous to the wearer, but capable of injecting antidotes within seconds and preferably also combines multiple means of sensing chemical and biological agents as well as monitoring user's physiological response to the antidotes thereby to enable tailored and continuous infusion of antidotes while minimizing the side effect of the antidotes themselves. A key embodiment of this invention is the incorporation of an electronically-controlled needle or cannula that will deploy only when commanded to. When not deployed, the device sits inertly against the patient's skin, affixed by adhesive and or elastic materials.

### BACKGROUND OF THE INVENTION

1. Field of the Invention

The present invention generally relates to radio-enabled devices for detection and remedy of biological and chemical warfare attack. More particularly, this invention relates to an on-body apparatus that provides a means of treatment which is innocuous to the wearer, but capable of injecting antidotes within seconds and preferably also combines multiple means of sensing chemical and biological agents as well as monitoring user's physiological response to the antidotes.

2. Description of Related Art

Apparatus for detecting chemical and biological agents is known in the prior art. More specifically, U.S. Pat. No. 7,977,113 to Donaldson et al. discloses a biological agent detector method. The biological agent detector detects the presence of any biological agents, such as anthrax or other biological warfare agents in a sample of air. The biological agent detector includes a bio-concentrator that concentrates an aerosol and a pyrolyzer portion including two detecting devices. One device operates in an aerosol sample collection mode and the other operates in a sample analysis mode. The sample collected is heated to form pyrolysis products which are transported to a mass spectrometer for identification and analysis.

U.S. Pat. No. 7,294,466 to McMillan relates to a multiplexed detection method using real time polymerase chain reaction (PCR) for detection of biological agents in a sample, e.g. bacteria, viruses, and biological toxins. The method utilizes two markers for each agent; the presence or absence in a sample of each of the two markers per agent is determined in separate reaction.

U.S. Pat. No. 7,405,073 to Tilles et al. is also directed to a method using PCR technology for identifying biological agents.

There exists commercially available handheld advanced nucleic acid analyzer (HANAA) in the marketplace using PCR technology for detection of biological agents.

U.S. Pat. No. 6,838,292 to Rajan et al. discloses methods based on a phosphorescence-emission detection system using chelate-stablized lanthanides for the detection of bacterial agents such as Bacillus anthracis and Clostridium botulinum.

Apparatus for detecting and treating chemical and biological agents is known in the prior art. More specifically, by way of example, U.S. Pat. No. 7,354,429 B2 to Sparks et al. discloses a device and method capable of sensing the presence of biochem agents, and delivering precise amounts of one or more antidotes to treat a victim exposed to the agents.

Although previous disclosures have dealt with the above sensing methods, a means of treatment that is innocuous to the wearer, but capable of injecting antidotes within seconds has not previously been described. Current technology relies upon providing personnel at risk with injector devices that must be self-administered. The ability to use such devices is dependent upon personnel recognizing the threat, reacting appropriately, and remaining conscious long enough to do so. Moreover, increasing use of body armor may make traditional injection devices impractical. While personnel could be equipped with an infusion pump with pre-inserted needle that maintains subcutaneous access via standard tubing and cannulas or needles, such apparatus is uncomfortable to wear in combat situations, is likely to meet with user resistance, and may be dislodged by the physical activity of combat.

Therefore, there exists a need for a light weight portable device which provides immediate first defense treatments for soldiers against chemical and biological terrorist attacks. Such a device would be comfortable to wear in combat situations and capable of deploying a needle or cannula that can pierces the skin upon activation by its controller and delivering said antidotes upon demand.

### SUMMARY OF THE INVENTION

One object of the present invention is to provide a device having antidotes delivery system capable of deploying a needle or cannula capable of piercing the skin upon activation by its controller and delivering said antidotes upon demand. The device is capable of delivering precise amounts of one or more antidotes to treat a victim.

Another object of the present invention is to provide a device having detectors/sensors capable of detecting chemical or biological agents.

Another object of the present invention is to provide a device having detectors/sensors capable of detecting soldiers' physiological response to the antidotes so that the antidote dose administration can be tailored according to the individual's physiological conditions.

A further object of the present invention is to provide a device having computer-based controller unit that maintains direct control over the injection device.

The device of the present invention in general comprises three modules: a detecting module including at least one sensor capable of detecting at least one chemical and/or biological warfare agent and/or one sensor capable of monitoring a person's physiological responses to antidote(s) countering the agents; a delivery module which is capable of delivering at least one antidote counteracting biological and/or chemical agents and/or depending on soldier's physiological responses to the antidote(s); and a controller module which controls connection via wired and/or near-field and/or long range radio wireless communication between the delivery module, the detecting module, a central control, and a remote monitoring stations, as well as controls delivery of antidotes. The at least one sensor for detecting chemical and/or biological agents included in the detecting module may be physically coupled to the delivery and controller modules and worn on a person or separate from the delivery and controller modules and not worn on a person.

The present invention will utilize technologies capable of detecting biological agents and/or chemical agents to fabricate the detecting module. Such technologies are well known to those with ordinary skill in the arts including but not limited to mass spectroscopy which uses ion mass spectral fingerprint or specific molecular ion masses unique to agents, UV/FTIR/VIS spectroscopy which uses absorption/fluorescence/emission spectral wavelength fingerprint unique to agent, physical property such as unique conductivity, density, polarity, and chemical property which uses chemical reactivity of agent and bio-reactivity of agent (including PCR). In the embodiments of the present invention that are intended for the soldiers to wear in the battlefield, membrane based light weight portable sensors utilizing aforementioned technologies are desired. The technology related to sensors monitoring user's physiological conditions such as heart beat (pulse), blood pressure, skin moisture (sweat), body temperature are also well known to those with ordinary skill in the art.

These sensors may be on-person or may be deployed at the squad level (i.e. located within proximity of the device wearer), or at the combat theater level (i.e. located elsewhere in the theater of operation). Some threats must ideally be detected before they reach the person under attack and sensors for those threats might be deployed on remote terrestrial, airborne, or even orbital platforms. Other sensors might be deployed on personnel other than the wearer of the device, such as on a combat medic or forward observer. In all cases, however, the sensor(s) must connect with a computer-based controller unit via wired or radio communications and such communications may be near-field or long-range in nature and that controller maintains direct control over the antidotes delivery module. The sensor(s) are capable of detecting and identifying multiple different chemical and biological warfare agents in a sample (e.g. air and water) surrounding a person.

The controller module has capability for both near-field and long-range radio communication. It may be controllable both by the wearer and/or under central control using wireless communication. The controller unit triggers the delivery device based on the input of a single sensor or via an algorithm that interpolates among a plurality of sensors. The controller unit utilizes algorithms based on the personal characteristics of the wearer in order to deliver an individualized dose of antidote(s) in a controlled manner. The sensors are used to inform the algorithm of the physiological response to the antidote(s) and thereby to enable a continuous infusion of antidote(s) that maximizes neutralization of the attacking agent, while minimizing side effects of the antidote(s) themselves. The controller unit may also be overridden by central command and by the wearer.

The controller module contains computer algorithms for insuring that false signals and random sensing (i.e. false positive signals) are not able to trigger the injection device (means) of the antidotes delivery module.

The delivery module is designed to be lightweight and small so that a soldier can carry in the battlefield. The delivery system may include at least one reservoirs separately storing one antidote counteracting biological and chemical agents; means for transporting antidotes from antidotes reservoirs to injection means; means for injection (e.g. needle, cannula) to penetrate soldier's skin and delivering antidotes into soldier's body; means for triggering injection; and means for communication between the selecting means and the delivering means. The detectors/sensors preferably communicate with the controller and identify the antidote(s) capable of counteracting the detected agent for the delivery device. In turn, the controller communicates with delivery module to start delivery of antidote(s). Some aforementioned components may be combined in different embodiments to serve the same combined function. The delivery module is operable to select and deliver the at least one antidote intravenously, subcutaneously, or intramuscularly etc.

A key embodiment of this invention is the incorporation of an electronically-controlled needle or cannula that will deploy only when commanded to. When not deployed, the device sits inertly against the patient's skin, affixed by adhesive and or elastic materials.

The detector/sensor, delivery and controller modules may be miniaturized and combined to become one portable unit capable of immediate treatment against biological and chemical attack where the medical response time makes a big difference. When these three modules are combined together, the unit will have the capabilities to detect the type and amount of biological or chemical agent present, and quickly communicate the information to the controller which in turn quickly controls the selection and precise delivery of appropriate amounts and concentrations of the appropriate antidote(s) through the antidotes delivery module into the soldier's body after the injector means pierces through the soldier's skin

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

FIG. 1 is a schematic of a device capable of delivering antidote(s) counteracting chemical or biological agent(s) in accordance with the present invention.

FIG. 2 is a schematic of a device capable of detecting chemical or biological agent(s) and delivering antidote(s) counteracting chemical or biological agent(s) in accordance with the present invention.

FIG. 3 is a schematic of a device capable of detecting chemical or biological agent(s) and monitoring user's physiological conditions and delivering antidote(s) counteracting chemical or biological agent(s) adjusted base on user's physiological conditions according to the present invention.

### DESCRIPTION OF THE PREFERRED EMBODIMENT

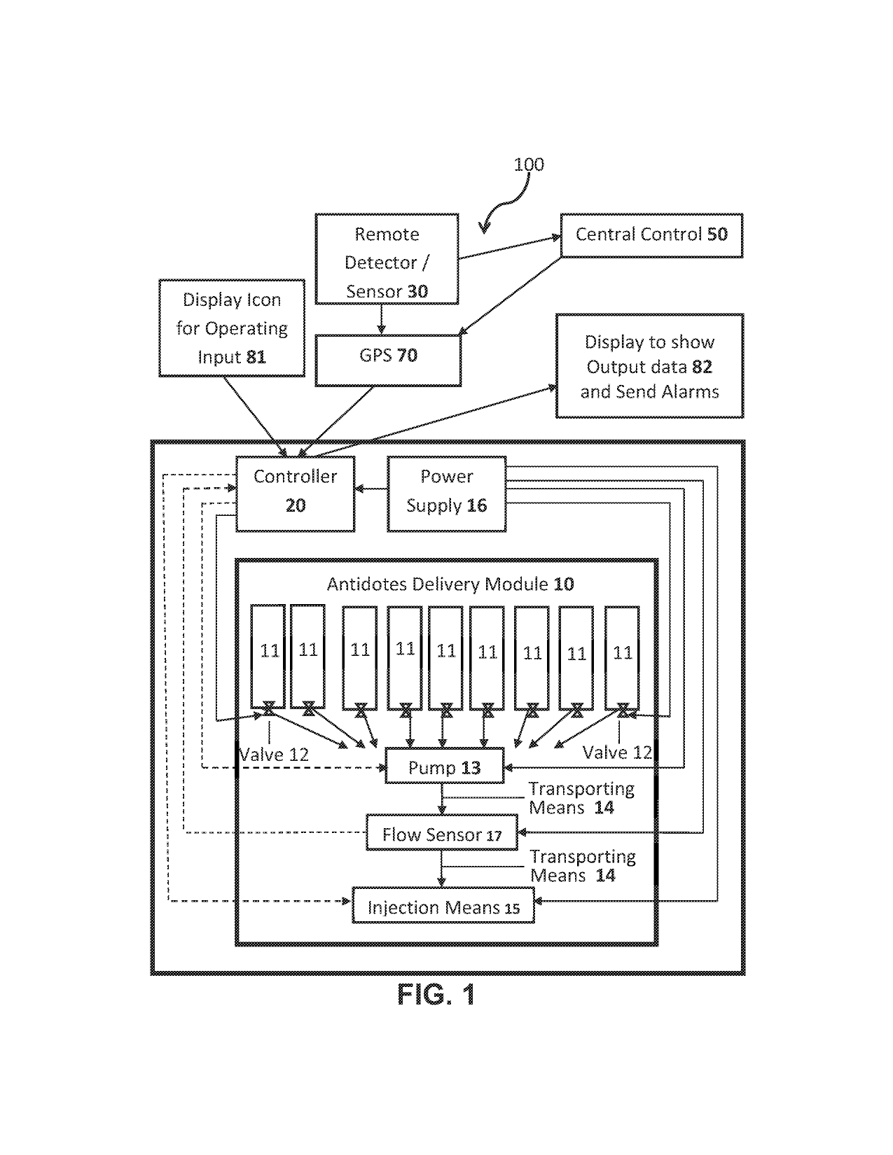

Referring to FIG. 1, there is disclosed a device capable of delivering antidote(s) counteracting chemical or biological agent(s) in accordance with the present invention. The system 100 comprises an antidote(s) delivery module 10 and a computer based controller 20. The antidotes delivery module 10 comprises one or more reservoirs 11 separately storing antidotes counteracting biological and chemical agent; a valve 12 connecting to each reservoir 11 selectively opens and closes upon command received from controller 20; a pump 13 driving the antidotes from reservoir 11 through transporting means 14 (a tubing in this embodiment) to the injection means 15 (e.g. needles or cannulas). In other embodiment, the reservoir 11 and transporting means 14 may be combined in one unit as a cartridge which stores and transports antidotes to the injection means 15.

This electronically controlled injection means 15 must be waterproof and connected to the body in such a manner that it can withstand vigorous activity. When worn in normal circumstances, the injection means 15 (e.g. needle or cannula), sitting inertly against the user's skin, affixed by adhesive and or elastic materials, does not pierce the skin and does not cause any discomfort to the wearer. The injections means may also stay inside a cartridge in normal circumstances and sticks out of the cartridge to penetrate the user's skin and deliver the antidotes into the user's body when triggered by controller's command.

When a biological or chemical agent is detected by detectors/sensors at remote sites 30, the detectors/sensors transmit data to the controller 20 or central control 50 via radio communications and such communications may be near-field or long-range in nature, which in turn sends a command to the antidotes delivery module 10. Upon receiving command from the controller 20 (e.g. an electronic signal), the injection means 15 pierces soldier's skin; the valve 12 opens the reservoir 11 which stores the antidotes capable of counteracting the detected agent; the pump 13 starts to drive/withdraw the antidote out of the reservoir 11 and transport to the injection means 15 to treat the soldier who has been exposed to the biological or chemical agents detected by the sensors 30. The antidotes delivery module 10 further comprises a power supply 16 which may be a battery for powering valves 12, pumps 13, and injection means 15.

The antidote delivery module 10 may further comprise a flow sensor 17 to measure the flow of the antidotes which in terms can be converted into dose per time unit that has been injected into victim's body. Most of the time if the pump 13 accurately and precisely operates according to the command received from the controller 20, the flow sensor 17 may not be necessary. However, the flow sensor 17 provides additional check-up when the antidote is potent and only a small quantity is needed.

The controller module 20 with capability for both near-field and long-range radio communication may be controllable both by the wearer and/or under central control 50 using wireless communication. The controller module 20 communicates in wire and/or wireless with antidotes delivery module 10, and detectors/sensors at remote monitoring sites 30 through global positioning satellite (GPS) 70. Other wireless technologies known to those with ordinary skill in the art may be used for the intended purpose. The controller module 20 maintains direct control over the antidotes delivery module 10 including triggering injection means 15 based on the input of a single sensor or via an algorithm that interpolates among a plurality of sensors. The controller 20 selects which valve(s) 12 of reservoir(s) 11 to open and how much dose to deliver in response to the input provided by the detector/sensor 30 and controls the valve 12 and pump 13 to deliver the antidote(s) from the reservoir(s) 11 through the opened valve(s) 12 and the tubing 14, reaching the injection means 15, and actuates injection mean 15 to penetrate soldier's skin when treatment is required in response to detection of a harmful agent. When sufficient antidote(s) have been delivered for the type and amount of agent(s) detected the controller 20 sends out commands to close the valve 12, stop the pump 13, and terminate the treatment. The controller module 20 contains computer algorithms for ensuring that false signals and random sensing (i.e. false positive signals) are not able to trigger the antidotes delivery module 10 including the injection device (means) 15.

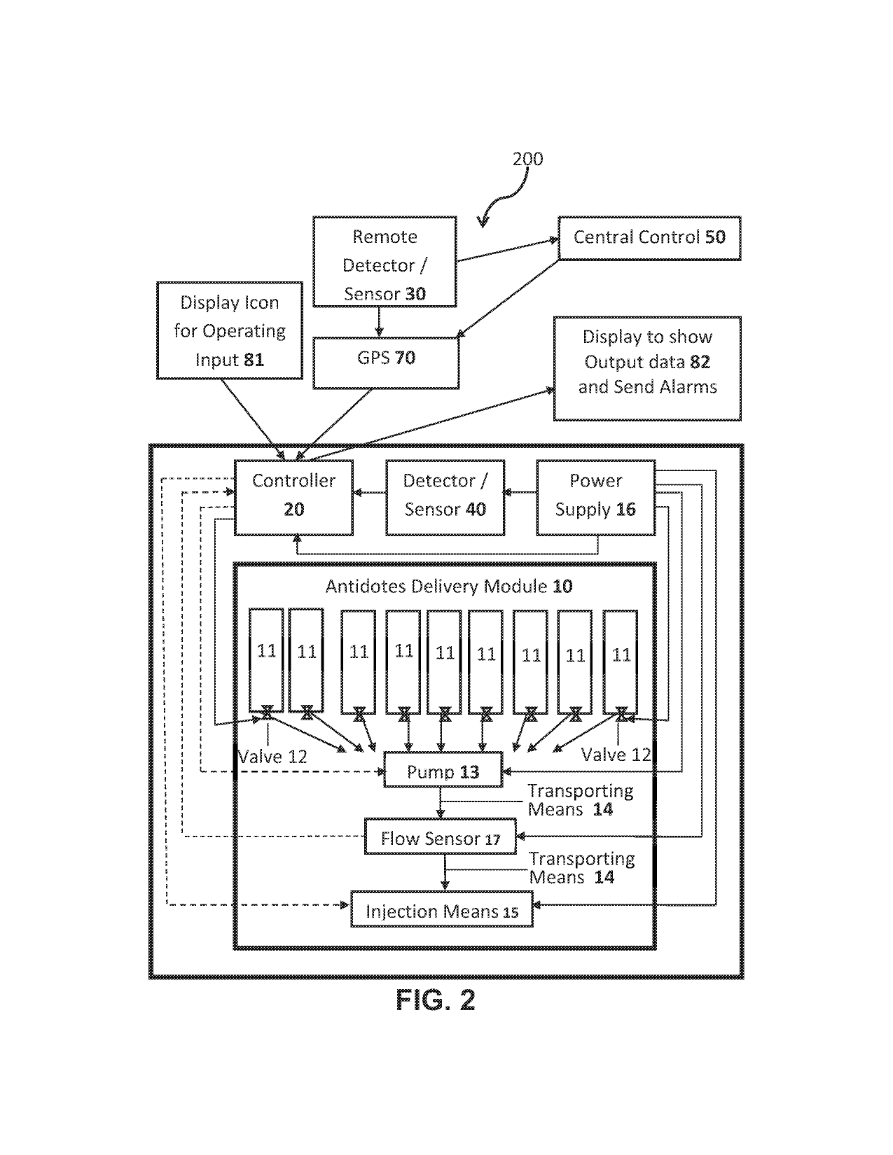

The antidotes delivery module 10 and controller 20 according to the present invention can be physically separate from the detectors/sensors for detecting the biological and chemical agents. As previously discussed, the detectors/sensors for detecting the biological and chemical agents can be carried by a soldier or a combat medic or forward observer or remotely deployed at the squad level or at the combat theater level. In all cases, the detectors/sensors must communicate with a computer-based controller unit 20 that maintains direct control over the antidotes delivery module 10 including injection means. For the detectors/sensors carried by a soldier, they can be physically coupled to antidote delivery module 10 and controller module 20 in one unit as illustrated in FIG. 2. System 200 comprises an antidotes delivery module 10 capable of delivering multiple antidotes at the same time when multiple biological and/or chemical threats are detected, detector/sensor 30, 40 capable of detecting biological and chemical agents as well as controller module 20 to communicate with remote detector 30 and integrated detector/sensor module 40 and control delivery module 10 such that the delivery module can immediately deliver antidotes through multiple-channels at the same time after the detector/sensor module 40 detects biological/chemical threats. The delivery module 10 comprises a plurality of reservoirs having valves for each reservoir to control flow through and at least one pump to withdraw the flow from the selected reservoir(s) through opened valve(s). Different mechanisms to drive/withdraw and transport the antidotes from reservoirs 11 to the injection means 15 may be implemented in other embodiments. As such, the embodiment of the present invention is capable of detecting the type and amount of biological and/or chemical agents present, and quickly and precisely delivering the appropriate amounts and concentrations of the appropriate antidote(s) to the victims; and thus is a rapid-response unit suitable for use as the first defense for individuals against chemical and biological attack.

The technologies to fabricate detectors/sensors for detecting the biological and/or chemical agents are well known to those with ordinary skill in the art. Any suitable technologies mentioned in the sections of background and summary may be adopted to fabricate the detector/sensor module of the present invention. Thus, no detailed description will be provided here.

One of the challenges of modern chemical and biological warfare is that the biological effect of the warfare agent is frequently longer in duration than the biological effect of the antidote. Prior art is based upon bolus infusion of antidote(s). However, the kinetics of the antidote are frequently such that administration in a single bolus in order to achieve sufficient duration of effect leads to unwanted side effects. For instance the amount of atropine that must be delivered by bolus injection to overcome modern chemical agents may lead to blurred vision, dry mouth, and other side effects that will impair combat effectiveness. In such situations, personnel may resist administering effective treatment because of the risk of rendering themselves combat-ineffective.

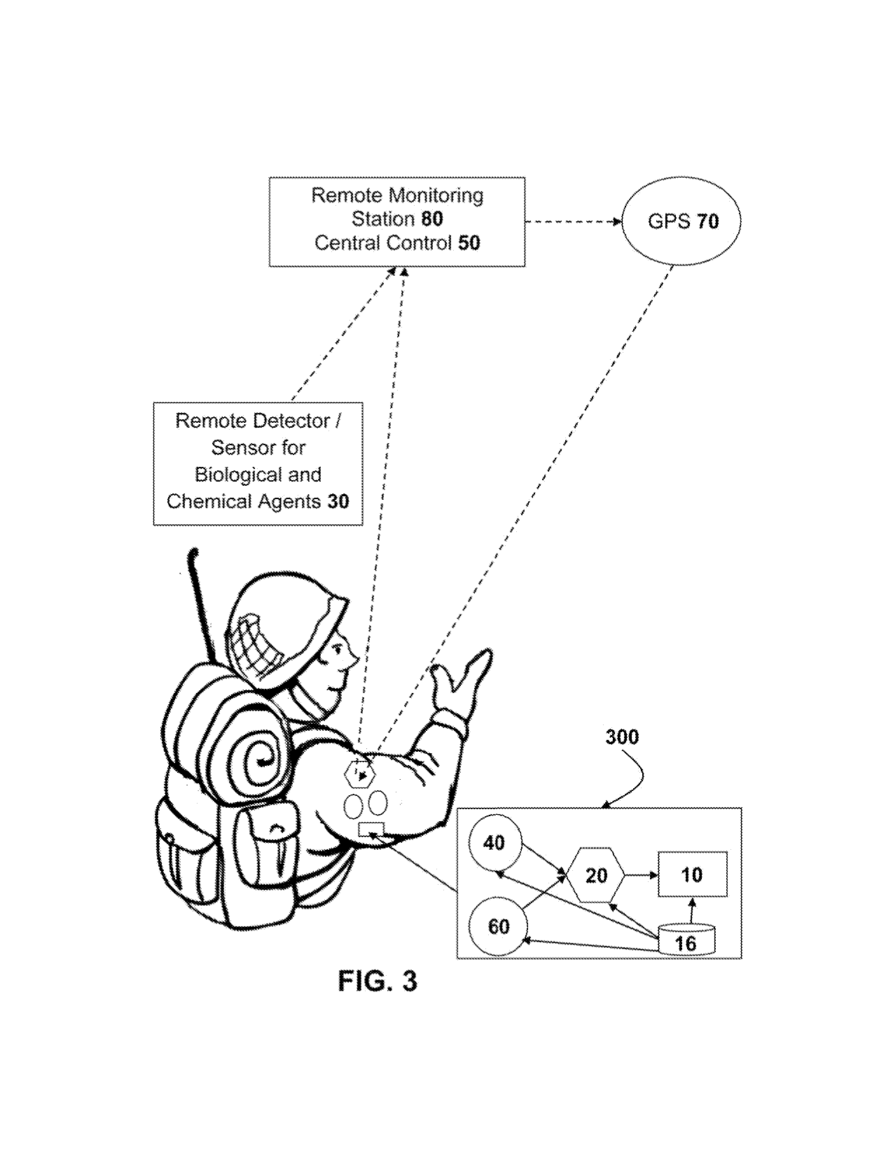

Prior art is also based on the assumption that the dose of the antidote(s) must be predetermined, rather than tailored to the physiological response. In the preferred embodiment of the invention that is depicted in FIG. 3, the controller module 20 in system 300 is in wireless communication through global positioning satellite (GPS) 70 with a remote monitoring station 80. Not only can the algorithm of the controller module 20 alter the infusion rate of antidote(s) by controlling the rate of delivery in the delivery module 10, medical personnel in the monitoring station 80 can override said algorithm and tailor the administration of antidote(s) based on the presence and concentration of the biologic and chemical agents collected by detectors/sensors from remote site 30 and/or carried by on-site personnel 40, as well as physiologic conditions of the soldiers collected by additional sensors 60 attached to the soldiers. Medical personnel in the monitoring station 80 overrides said algorithm and tailor the administration of antidote(s) through controller 20; the controller 20 receives data from the monitoring station 80 and provides signals to the drug delivery module 10 for adjusting the amounts of the drug to be delivered to the body. Alternatively, in other embodiment, the monitoring station 80 can directly control the delivery module 10 without going through controller 20. In summary, the controller unit utilizes algorithms based on the personal characteristics of the wearer in order to deliver an individualized dose of antidote(s) in a controlled manner. The sensors are used to inform the algorithm of the physiological response to the antidote(s) and thereby to enable a continuous infusion of antidote(s) that maximizes neutralization of the attacking agent, while minimizing side effects of the antidote(s) themselves. The controller unit may also be overridden by central command and by the wearer. The remote monitoring station 80 may act as the central control 50 but the central control 50 may not have medical professionals to monitor the user's physiological conditions.

Such additional sensors 60 monitoring physiological responses to the antidote(s) includes heart beat (pulse), skin temperature, skin moisture, blood pressure, and other biologic functions known to be related to common warfare agents, such as cholinergics. The additional sensors 60 may be physically coupled to delivery module and controller module in one unit as demonstrated in FIG. 3. Alternatively, the additional sensors 60 may be physically separate from delivery and controller module so that the additional sensors 60 can closely contact with soldier's body on appropriate locations. The algorithms for acting upon information from the sensors 60 would additionally be informed by pre-programmed information on weight, body fat composition, and other metabolic variables. The system 300 also comprises a power supply (e.g. battery) 16 for powering these modules. Similar to system 200 and 100, the controller 20 communicates with integrated detector/sensor module 40, remote detector/sensor 30, delivery module 10, and additional sensor 60 via electrical wire and/or wireless (e.g. radio frequency).

Referring back to FIG. 1 and FIG. 2, the antidotes delivery module 10 is equipped with sufficient antidotes reservoirs 11 to treat the following chemical and biological agents that have antidotes and pose a serious threat in the hands of terrorists: sarin, VX, tabun, soman (nerve agents); cyanide (blood asphyxiants); lewisite (blister); anthrax, brucellosis, plague, Q fever (bacterial pathogen); and botulinum (biological toxin). As shown in FIGS. 1 and 2, the antidotes delivery module 10 has nine reservoirs 11 that can store atropine and 2-PAM-CI for nerve agents, botulinum antitoxin, antibiotics (e.g. penicillin, doxycycline and ciprofloxacin) for the initial treatment of bacterial pathogen (e.g. anthrax, brucellosis, Q-fever, plague), BAL or dimercaprol for lewisite and sodium nitrile followed by sodium thiosulphate for cyanide poisoning. Subsets of these drug combinations or alternate drugs could also be utilized in the antidote delivery module 10. A different number of antidotes reservoirs 11 may be fabricated in other embodiments.

Furthermore, the device of the present invention may comprise display icons 80 allowing user to make operating inputs 81 to the controller 40 and display data output 82 from the controller 40 to enable the operation of the device to be observed and controlled by the user. The operating input may be made through physical buttons in other embodiment. The device may further sends an alarm to alert soldiers and remote sites once the detector/sensor module 40 detects biological and chemical threats. The device further comprises means for locating the person.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

What is claimed is:

1. A device for detecting chemical and biological agent(s) and treating a person who is exposed to chemical or biological agent(s) comprising a detecting module including at least one sensor capable of detecting at least one chemical or biological agent; a delivery module for delivering at least one antidotes to treat the person who is exposed to chemical and/or biological agent(s); and a controller module having capability of connecting with detecting module and delivery module via wired communication and via both near-field and long-range radio communication, the controller module triggers the delivery module based on the input from the detecting module.

2. The device of claim 1, wherein the sensor is physically coupled to the delivery module and controller module and carried on the person.

3. The device of claim 1, wherein the sensor is remote from the delivery module and controller module and not carried on the person.

4. The device of claim 1 further comprises at least one sensor worn on a person for monitoring the person's physiological response to the antidote(s) and thereby to enable a tailored continuous infusion of antidote(s) that maximizes neutralization of the attacking agent, while minimizing side effects of the antidote(s) themselves.

5. The device of claim 4, wherein, the activation of the device and monitoring of the physiologic effects of the antidote(s) is under the surveillance of a remote monitoring station wherein medical personnel are able to tailor the device's function to the exigencies of the battlefield and the individual physiologic response of the wearer.

6. The device of claim 1, wherein the controller module utilizes algorithms based on the personal characteristics of the wearer in order to deliver an individualized dose of antidote(s) in a controlled manner.

7. The device of claim 1, wherein the controller module may be controllable by the wearer and under central control using wireless communication, the controller module may be overridden by central command and by the wearer.

8. The device of claim 1, wherein the delivery module includes at least one reservoir of at least one antidote, an injection means that mechanically penetrates the skin upon receiving an electrical signal, a pump that causes at least one antidote to be injected in a precise dose via the injection means, and a communication means to the controller module.

9. The device of claim 8, wherein the injection means is automatically deployed to pierce the wearer's skin only upon electronic command.

10. The device of claim 1 further comprising sending an alert signal to the person and a remote site if any chemical or biological agent is detected.

11. The device of claim 1, wherein the detecting module is capable of detecting and identifying multiple different chemical and biological agents in a sample.

12. The device of claim 11, wherein the controller is capable of identifying multiple antidotes to counteract the multiple different agents.

13. A method of detecting chemical or biological agent(s) and treating a person who is exposed to the agent(s) comprising the steps of: detecting the presence and identifying chemical or biological agent using a detecting module including at least one sensor capable of detecting and identifying at least one agent selected from the group consisting of chemical and biological agents; selecting at least one antidote as being capable of counteracting the identified agent(s) by a controller module; and delivering at least one antidote as being capable of counteracting the identified agent(s) to the person by a delivery module.

14. The method of claim 13, wherein the steps of delivering at least one antidote as being capable of countering the identified agent(s) to the person comprises the steps of: receiving a signal from the controller module, causing the delivery module to actuate the insertion of an injection means into the skin, and then causing the delivery unit to deliver a metered dose of at least one antidotes.

15. The method of claim 13, wherein the step of detecting the chemical or biological agent(s) is performed by the sensor coupled to the delivery and controller modules and carried on the person.

16. The method of claim 13, wherein the step of detecting the chemical or biological agent(s) is performed by the sensor remote from the delivery and controller modules and not carried on the person.

17. The method of claim 13 further comprising electronically-controlled automatic deployment of the delivery module to deliver antidote(s) into the body of the person after the step of detecting the agent(s).

18. The method of claim 13 further comprising monitoring the person's physiological responses to the antidote(s) and thereby to enable tailored continuous infusion of the antidote(s) while minimizing the side effect of the antidote(s) themselves.

19. A device for detecting a chemical or biological agent and treating a person who is exposed to a chemical or biological agent comprising a detecting module including at least one sensor capable of detecting biological and chemical agent(s); a delivery module; and a controller module with capability for both near-field and long-range radio communication; wherein the sensor(s) being physically coupled to or remote from the delivery and controller modules is connected to the controller module via wired or radio communications and such communications may be near-field or long-range, the controller unit may be controllable both by the person and/or under central control using wireless communication, the controller unit triggers the delivery device based on the input of a single sensor or via an algorithm that interpolates among a plurality of sensors, the controller unit utilizes algorithms based on the personal characteristics of the person in order to deliver an individualized dose of antidote(s) in a controlled manner, the controller unit may also be overridden by central command and by the person, the delivery device includes at least one reservoir of at least one antidote, an injection means that mechanically penetrates the skin upon receiving an electrical signal, a pump that causes at least one antidote to be injected in a precise dose via the injection means, and a wired or wireless communication to a controller unit.

20. The device of claim 19 further comprising sensors for monitoring physiological responses to the antidote(s) and thereby to enable a continuous infusion of antidote(s) that maximizes neutralization of the attacking agent, while minimizing side effects of the antidote(s) themselves.
